(19) United States
(12) Patent Application Publication
(10) Pub. No.: US 2026/0125643 A1
(43) Pub. Date: May 7, 2026

(54) SKIN ORGANOID, METHOD FOR PRODUCING SAME, AND METHOD FOR EVALUATING DRUG BY USING SAME

(71) Applicant: ORG CORP., Goyang-si Gyeonggi-do (KR)

(72) Inventor: Jaeyeaon CHO, Goyang-si, Gyeonggi-do (KR)

(73) Assignee: ORG CORP., Goyang-si Gyeonggi-do (KR)

(21) Appl. No.: 19/115,624

(22) PCT Filed: Sep. 27, 2023
(86) PCT No.: PCT/KR2023/015147

§ 371 Date: Mar. 26, 2025

(30) Foreign Application Priority Data
Sep. 27, 2022 (KR) 10-2022-0122635

(51) Int. Cl. C12N 5/071

(52) U.S. Cl. CPC C12N 5/0625; C12N 5/0627; C12N 2501/734; C12N 2513/00; C12N 2533/30; C12N 2533/90

(57) ABSTRACT

Provided is a method for producing a skin organoid, the method comprising: a step for performing a first culture of a primary skin cell in a first medium with at least one support among an extracellular matrix (ECM) and polyethylene glycol (PEG) to form a first skin organoid; and a step for performing a second culture of the first skin organoid in a second medium. In addition, the present disclosure provides: a skin organoid cultured by the method described above, the skin organoid having a round, spherical, three-dimensional shape and comprising a center portion, which is densely packed with cells, a stratum corneum formed on the outside of the center portion, and a hair root, wherein the cells can move from the center portion to the stratum corneum; and a method for evaluating drug toxicity by using same.

### TECHNICAL FIELD

The present disclosure relates to a skin organoid having a structure similar to living skin, and more particularly, to a skin organoid having a round, spherical three-dimensional shape and including an outer stratum corneum, a central portion densely packed with cells, and a hair root, in which the cells move from the central portion to the stratum corneum, a method for producing the same, and a method for evaluating a drug using the same.

### BACKGROUND ART

[Department Name] Gyeonggido Economic Science Promotion Agency

[Research Project Name] Research and Development Support Project for Women Scientists and Engineers

[Research Subject Name] Development of Skin Organoid Production Platform Using Human Dermal Extracellular Matrix

[Research Period] Jul. 1, 2023 to Jun. 30, 2024

In the early stages of new drug development, there is a need for a model for evaluating exact toxicity and efficacy prediction. With current technology, animal models may most closely simulate the toxicity and efficacy of new drugs. However, animal experiments are burdensome in time and money, and it is difficult to completely reflect an in vivo environment of the human due to differences in genetic, biochemical and metabolic processes between species. In addition, it is difficult to technically monitor a process happening inside animals and it may also be ethically problematic.

Accordingly, although primary cultured cells which are directly isolated from human tissues and cultured in vitro are used as a standard model, it is difficult to obtain tissues, and there are experimental limitations in that tissue cells do not expand in vitro. Furthermore, since a two-dimensional cell-based in vitro model is more efficient than the primary cultured cells derived from human tissues in terms of cost and labor, the model is widely used for evaluating drug toxicity and efficacy. However, the two-dimensional cell-based in vitro model is insufficient to embody the physiological functions and tissue complexity resulting from cell-cell and cell-extracellular matrix interactions.

Meanwhile, an organoid is attracting attention as a new biomimetic model. The organoid is formed into a three-dimensional structure such as organs by growing stem cells into specific cells. Unlike the two-dimensional cell-based in vitro model, the organoid may be cultured in a three-dimensional environment and cultured for a longer period of time. In addition, the organoid is small in size, but constituent cells and structures thereof are similar to those of a real organ. Accordingly, the organoid has been evaluated as an optimal specimen to determine the efficacy and stability of the drug in the process of developing new drugs. Furthermore, organoid-related fields have high potential to be used not only for evaluating the drug toxicity and efficacy of new drug development, but also for disease models, cancer research, personalized medicine, regenerative therapeutics, and the like.

To date, various organoids, such as stomach, intestine, early liver, thyroid, lung, brain, and the like have been successfully developed. However, a skin organoid developed to date exhibits a structurally different characteristic from a biological skin. For example, the skin organoid differs morphologically and functionally from the biological skin, as a stratum corneum and an epidermal layer are generated inside the organoid. Accordingly, a conventional skin organoid is not used as an organoid itself, but is chopped and reused as a cell-like structure, and thus has limitations in representing a biological skin in evaluation of drug and toxicity as the organoid.

Therefore, there is a need to develop a skin organoid in which a stratum corneum and an epidermal layer are formed on the outside to be mimicked morphologically and functionally similar to a biological skin.

The background art of the invention has been prepared to more facilitate understanding of the present disclosure. It should not be understood that the matters described in the background art of the invention exist as prior arts.

### DETAILED DESCRIPTION OF THE INVENTION

### Technical Problem

More specifically, since a conventional skin organoid has a structure in which the epidermis including a stratum corneum is located inside the tissue, unlike a biological skin, the skin organoid has been used by exposing the inside of the tissue to the outside by chopping and simply layering cells.

In addition, since the conventional skin organoid does not contain biological skin component cells such as vascular cells and immune cells, the skin organoid does not represent a biological skin even physiologically and functionally. Accordingly, the conventional skin organoid has been used only for a simple skin irritation test, and has not been used for evaluating material efficacy such as drug and toxicity tests and evaluating skin regeneration.

Furthermore, the conventional skin organoid easily has a short culture period (expiration date) within a week due to the structural limitations described above, and thus limits the establishment of experimental plans and the reproduction of results, thereby making long-term tracking observation impossible.

Meanwhile, the present inventors noted that a biological skin included various heterogeneous cells. More specifically, the present inventors noted that the biological skin consisted of the epidermis, dermis, and subcutaneous fat layer, which had different components depending on each characteristic. Furthermore, the present inventors recognized that the formation of various components of a biological skin had limitations in stem cells in a limited culture environment.

Accordingly, the present inventors discovered that in the case of using heterogeneous primary skin cells, rather than stem cells that differentiated into a specific system, it was easier to differentiate into skin cell components such as the epidermis and dermis.

Accordingly, the present inventors have developed a skin organoid that is structurally and functionally (physiologically) similar to a biological skin from heterogeneous primary skin cells, and a method for producing the same.

Therefore, an object of the present disclosure to be solved is to provide a method for producing a skin organoid under specific environments and conditions, which can culture primary skin cells to have a structure and function similar to a biological skin.

Another object of the present disclosure to be solved is to provide a skin organoid having an improved mimic degree of a biological skin which is produced by the production method described above and includes a stratum corneum on the outside and a hair root, and a method for evaluating a drug using the skin organoid.

The objects of the present disclosure are not limited to the aforementioned objects, and other objects, which are not mentioned above, will be apparent to those skilled in the art from the following description.

### Means for Solving the Problem

In order to solve the aforementioned object, an aspect of the present disclosure provides a skin organoid including a center portion (core) which is densely packed with cells, a stratum corneum formed on the outside of the center portion, and a hair root, in which the cells can move from the center portion to the stratum corneum, and having a round, spherical, three-dimensional shape.

According to a feature of the present disclosure, the stratum corneum may have a thickness of 1 to 100 μm or less, but is not limited thereto.

According to another feature of the present disclosure, the stratum corneum may include at least one of KRT10, KRT14 and loricrin, but is not limited thereto.

According to yet another feature of the present disclosure, the center portion may have a diameter of 100 to 1000 μm, but is not limited thereto.

According to yet another feature of the present disclosure, the center portion may include vimentin (VIM), but is not limited thereto.

Another aspect of the present disclosure provides a method for producing a skin organoid including a stratum corneum formed on the outside and a hair root, the method including a step of performing a first culture of skin cells (primary skin cells) in a first medium with at least one support of an extracellular matrix (ECM) or polyethylene glycol (PEG) to form a first skin organoid; and a step of performing a second culture of the first skin organoid in a second medium.

According to a feature of the present disclosure, before the performing a first culture, the method may further include a step of chopping a skin tissue isolated from a subject, and a step of obtaining heterogeneous skin cells (primary skin cells) by treating the chopped skin tissue with collagenase.

According to another feature of the present disclosure, the collagenase may include at least one of type I collagenase and type IV collagenase, but is not limited thereto.

According to yet another feature of the present disclosure, the obtaining heterogeneous skin cells may further include a step of culturing the primary skin cells to expand.

According to yet another feature of the present disclosure, the skin cell may have a size of about 100 μm or less, but is not limited thereto.

According to yet another feature of the present disclosure, the skin cell may include at least one of APOE, COL1A1, CD34, KRT7, KRT10, KRT19, HS3ST6, PECAM1 and VIM, but not limited thereto.

According to yet another feature of the present disclosure, the ECM may include at least one of a patch-shaped ECM and a fiber-shaped ECM, but is not limited thereto.

According to yet another feature of the present disclosure, the fiber-shaped ECM may be obtained by treating the patch-shaped ECM with a protein digestive enzyme.

According to yet another feature of the present disclosure, the fiber-shaped ECM may include at least one of collagen alpha-1(I) chain, collagen alpha-3(VI) chain, collagen alpha-2(I) chain, collagen alpha-2(VI) chain, collagen alpha-1(VI) chain, keratin type I cytoskeletal 9, keratin type II cytoskeletal 1, keratin type I cytoskeletal 10, biglycan, decorin, lumican and collagen alpha-1(III) chain, but is not limited thereto.

According to yet another feature of the present disclosure, the ECM may be obtained by culturing fibroblasts to form a fibroblast patch and decellularizing the fibroblast patch.

According to yet another feature of the present disclosure, the first culture may be performed for at least one period of about 1 hour to 3 days, but is not limited thereto.

According to yet another feature of the present disclosure, the first medium may include at least one of a B-27 supplement and a serum replacement, but is not limited thereto.

According to yet another feature of the present disclosure, the B-27 supplement may be contained in an amount of 1 to 10 v/v % with respect to the total volume of the first medium, but is not limited thereto.

According to yet another feature of the present disclosure, the serum replacement may be contained in an amount of 5 to 15 v/v % with respect to the total volume of the first medium, but is not limited thereto.

According to yet another feature of the present disclosure, the second culture may be performed for at least one period of about 7 days to 40 days, but is not limited thereto.

According to yet another feature of the present disclosure, the second medium may include at least one of the first medium, ascorbic acid, CHIRR99021, and a Wnt agonist, but is not limited thereto.

According to yet another feature of the present disclosure, the ascorbic acid may be contained in 2 to 500 mM based on 1 L of the second medium, but is not limited thereto.

Another aspect of the present disclosure provides a method for evaluating a drug using a skin organoid, including a step of treating the aforementioned skin organoid and a drug.

Hereinafter, the present disclosure will be described in more detail with reference to Examples. However, these Examples are only illustrative of the present disclosure, and thus it should not be interpreted that the scope of the present disclosure is limited by these Examples.

### Effect of the Invention

According to the present disclosure, it is possible to evaluate validation, that is, efficacy, side effects, and toxicity of a drug in new drug development by providing the skin organoid, the method for producing the same, and the method for evaluating the drug toxicity using the same.

More particularly, the present disclosure provides a skin organoid having a round, spherical, three-dimensional shape and including an outer stratum corneum corresponding to the epidermis of a biological skin, a center portion corresponding to the dermis, and a hair root, unlike conventional organoids that had a structure different from the biological skin, and not only has structurally the same layer formation as the biological skin, but also includes a physiologically similar cell cycle phenomenon to the biological skin. Accordingly, the skin organoid of the present disclosure may identify structural and/or physiological abnormalities of the skin for various substances.

Therefore, the skin organoid of the present disclosure may be used to evaluate side effects, toxicity and efficacy of a drug as a biomimetic model that is functionally and structurally similar to a biological skin.

Further, the skin organoid of the present disclosure may be used for screening of drug candidate substances in new drug development to dramatically reduce required cost and time, and may be used for physiological research and clinical trials of skin cancer. Furthermore, the present disclosure may be used for personalized diagnosis capable of reducing cost of treatment by allowing experiments on various causes of the present disclosure to prevent unnecessary drug administration.

The effects of the present disclosure are not limited by the foregoing, and other various effects are anticipated herein.

### BRIEF DESCRIPTION OF THE DRAWINGS

FIG. 1 illustrates a process of a method for producing a skin organoid according to one embodiment of the present disclosure.

FIG. 2A illustrates an exemplary diagram for a process of a step of obtaining skin cells in the method for producing the skin organoid according to one embodiment of the present disclosure.

FIG. 2B illustrates results of single cell transcription analysis for primary skin cells used in the method for producing the skin organoid according to one embodiment of the present disclosure.

FIG. 3 illustrates an exemplary diagram for a process of a first culturing step in the method for producing the skin organoid according to one embodiment of the present disclosure.

FIG. 4A illustrates an exemplary diagram for a process of forming ECM used in the method for producing the skin organoid according to one embodiment of the present disclosure.

FIG. 4B illustrates results of protein component analysis for fiber-shaped ECM used in the method for producing the skin organoid according to one embodiment of the present disclosure.

FIG. 5 illustrates microscopic images of a first skin organoid formed by the method for producing the skin organoid according to one embodiment of the present disclosure.

FIG. 6A illustrates staining images for LOR and KRT10 markers of the first skin organoid formed by the method for producing the skin organoid according to one embodiment of the present disclosure.

FIG. 6B illustrates staining images for a KRT14 marker of the first skin organoid formed by the method for producing the skin organoid according to one embodiment of the present disclosure.

FIG. 6C illustrates staining images for a VIM marker of the first skin organoid formed by the method for producing the skin organoid according to one embodiment of the present disclosure.

FIG. 7 illustrates microscopic images for a stratum corneum of the first skin organoid formed by the method for producing the skin organoid according to one embodiment of the present disclosure.

FIG. 8 illustrates microscopic images for the size of the first skin organoid formed by the method for producing the skin organoid according to one embodiment of the present disclosure.

FIG. 9 illustrates microscopic images for a second skin organoid formed by the method for producing the skin organoid according to one embodiment of the present disclosure.

FIG. 10 illustrates a flowchart for a method for evaluating a drug using a skin organoid according to one embodiment of the present disclosure.

### BEST MODE FOR CARRYING OUT THE INVENTION

Advantages and features of the present disclosure, and methods for accomplishing the same will be more clearly understood from embodiments described in detail below with reference to the accompanying drawings. However, the present disclosure is not limited to the embodiments set forth below, and will be embodied in various different forms. The embodiments are just for rendering the disclosure of the present disclosure complete and are set forth to provide a complete understanding of the scope of the invention to those skilled in the art to which the present disclosure pertains.

The term “or” as used herein means “and/or” unless otherwise stated.

The term “about” as used herein refers to a general error range for each value that is readily known to those skilled in the art. As used herein, a “about” value or parameter includes embodiments for the value or parameter itself. Furthermore, the term “about” indicates a range of values that are within 10% in any one direction (greater than or less than) of mentioned reference values, unless otherwise indicated or clear from the context.

The term “patient or subject” as used herein is used interchangeably and refers to any single animal, more preferably mammals (including non-human animals, such as cats, dogs, horses, rabbits, zoo animals, cattle, pigs, sheep, and non-human primates) in which treatment is required. The patient referred to in various embodiments of this specification may be a human.

As used herein, the term “differentiation” means that cells are developed at a level of a complex of specific cells or tissues or subjects having a specific function.

As used herein, the term “organoid” refers to a small embryoid body that reproduces both the shape and function of a tissue or organ. More specifically, the organoid needs to include one or more cell types among various types of cells constituting the organ or tissue, needs to be able to reproduce a specific function of each organ, and needs to be organized in a spatially similar shape to the organ by agglomerating cells. Such an organoid is different from a spheroid in that the organoid forms a system rather than a simple aggregate of cells, and may be used as patient-specific models for new drug development, artificial organs, disease therapeutics, and disease treatments.

As used herein, the term “medium” refers to a mixture for the growth and expansion of various cells in vitro, including essential elements for the cell growth and expansion, such as sugars, amino acids, various nutrients, serum, growth factors, minerals, and the like.

At this time, the term “extracellular matrix (ECM)” as used herein refers to a support in the development of a tissue having a three-dimensional structure which plays an important role in providing signals affecting various cellular metabolic pathways, such as expansion, differentiation and death of the cells. The ECM may store and supply biochemical factors required for the growth and differentiation of the cells, and provide physical environments which may be recognized by the cells at the same time. The ECM is a product produced by cells constituting each tissue as needed, and includes structural proteins such as collagen and elastin, polysaccharides such as glycosaminoglycan (GAG), adhesive proteins that help the adhesion of cells, and growth factors. Such an ECM consists of different components depending on a tissue and a cell to be derived, and has special physical properties.

### Example 1. Method for Producing Skin Organoid and Skin Organoid Produced Thereby

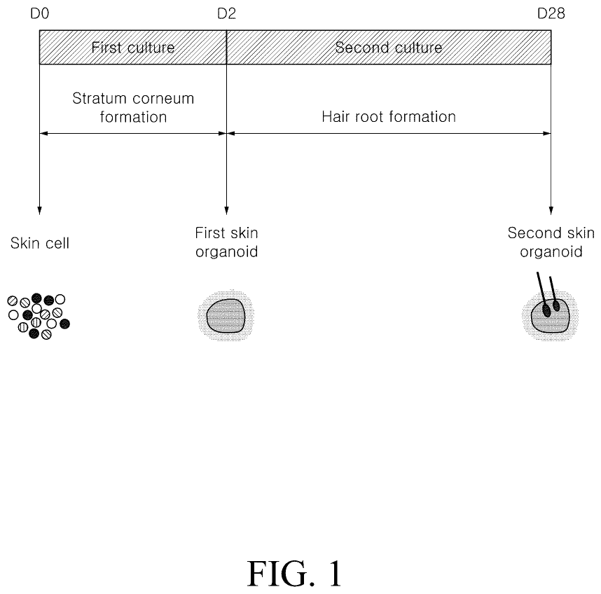

Hereinafter, a method for producing a skin organoid according to one embodiment of the present disclosure and a process for forming a skin organoid thereby will be described in detail with reference to FIGS. 1 to 6.

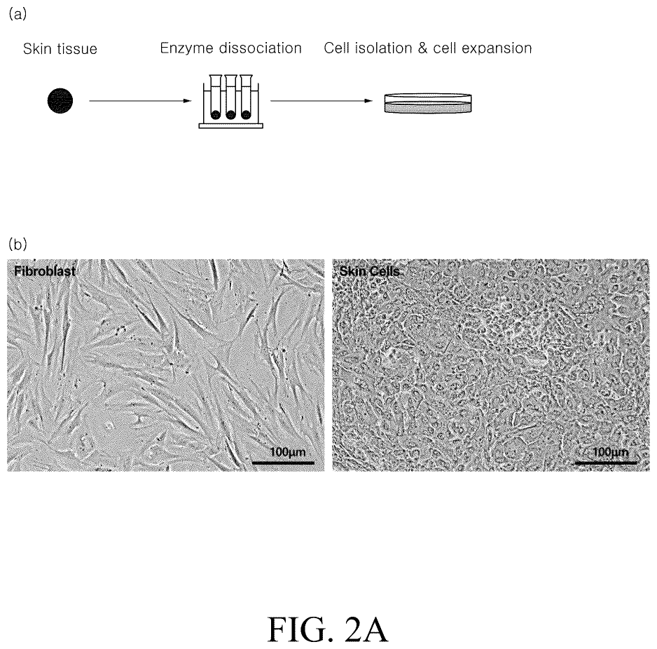

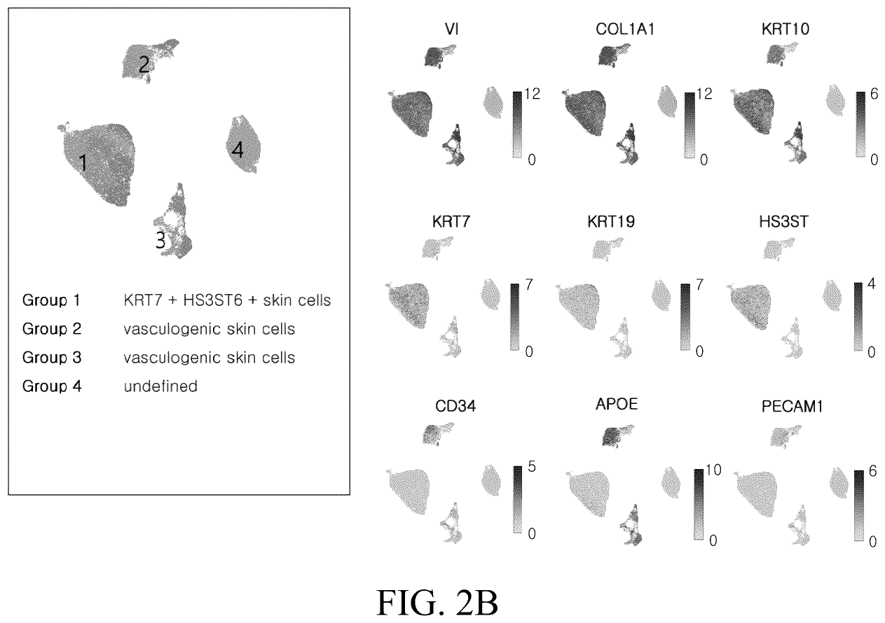

FIG. 1 illustrates exemplarily a process of a method for producing a skin organoid according to one embodiment of the present disclosure. At this time, for convenience of description, the process will be described with reference to FIGS. 2A to 6.

Referring to FIG. 1, the method for producing the skin organoid according to one embodiment of the present disclosure is a method for forming an organoid including a stratum corneum on the outside and a hair root, the method including a step of performing a first culture of skin cells in a first medium with at least one support of an extracellular matrix (ECM) or polyethylene glycol (PEG) to form a first skin organoid, and a step of performing a second culture of the first skin organoid in a second medium.

First, the skin cells used in the first culturing step may refer to primary skin cells obtained from a skin tissue isolated from a subject. For example, the skin cells used in the first culturing step may be obtained by dissociating the skin tissue into cell units using an enzymatic method and growing the cells in a culture container. Accordingly, the method for producing the skin organoid according to one embodiment of the present disclosure may include a step of obtaining the skin cells before the performing a first culture.

More specifically, referring to FIG. 2A, an exemplary diagram for a process of a step of obtaining skin cells in the method for producing the skin organoid according to one embodiment of the present disclosure is illustrated.

First, referring to (a) of FIG. 2A, the step of obtaining the skin cells may include a step of chopping or cutting a skin tissue (human skin biopsy) isolated from a subject, a step of dissociating the chopped tissue into cells using an enzymatic method (enzymatic dissociation), and a step of culturing the dissociated cells to expand.

The skin tissue isolated from the subject may be used all in various areas without restrictions, such as the face, arms, and the like of the subject.

The chopping in the chopping step may include any method capable of reducing the size of the tissue by applying a physical force, such as shear force, and may be performed using various instruments, such as a surgical knife or chopper.

The enzyme used in the dissociating step is collagenase, which may include at least one of type I collagenase and type IV collagenase, but is not limited thereto, and may include any substance capable of dissolving and decomposing (dissociating) a support substance such as collagen capable of connecting and supporting cells and/or tissues. For example, the enzyme used in the dissociating step may be used with dispase, protease, trypsin, and the like, as well as collagenase, but is not limited thereto.

The cells obtained in the dissociating step may be primary skin cells, which may be used directly for organoid formation. Accordingly, in the method for producing the skin organoid according to one embodiment of the present disclosure, the skin cells used for forming the organoid may be cells obtained through an enzymatic method, but are not limited thereto, and may be cultured and then used for uniform supply of cells.

Accordingly, in the culturing step, the primary skin cells obtained from the dissociating step may be cultured to expand in a medium containing 10% fetal bovine serum (FBS).

Finally, the method for producing the skin organoid according to one embodiment of the present disclosure may further include a step of chopping a skin tissue isolated from a subject, and a step of obtaining heterogeneous skin cells (primary skin cells) by treating the chopped skin tissue with collagenase before the step of producing the primary skin cells, which are material cells of the organoid, that is, the first culturing step, and further, the obtaining step may further include a step of culturing the primary skin cells to expand.

In this regard, referring to (b) of FIG. 2A, microscopic images of the primary skin cells obtained by the aforementioned process are shown.

The primary skin cells are shown to have a black peri-nucleus including the nucleus and a small size of about 100 μm or less, and the primary skin cells of the present disclosure are morphologically different from fibroblasts that have a spindle shape and the size of about 100 μm or more.

Furthermore, the primary skin cells of the present disclosure may be heterogeneous, unlike fibroblasts and specific-system stem cells generally used for organoid production.

More specifically, referring to FIG. 2B, results of single cell transcription analysis for primary skin cells used in the method for producing the skin organoid according to one embodiment of the present disclosure are shown.

The primary skin cells of the present disclosure may be divided into a total of four groups (group 1, group 2, group 3, and group 4), and group 1 is shown as skin cells (KRT7+ HS3ST6+ skin cells) that specifically express KRT7 and HS3ST6 associated with the components of the epidermal layer and the stratum corneum of the skin, and groups 2 and 3 are shown as vasculogenic skin cells.

At this time, since the cells for groups 2 and 3 are vasculogenic skin cells, the cells may form blood vessels within the skin in the organoid during cell growth.

Vimentin (VIM) is a marker of fibroblasts, i.e. dermal fibroblasts inside the skin, and is a structural protein involved in a cytoskeleton, a cell support, and the like. VIM is shown to be overexpressed in all groups 1, 2, 3 and 4, which may mean that cells in all groups may produce structural proteins such as vimentin and resulting cytokeratin.

COL1A1 is a marker expressed in keratinocytes and is included in various parts of the body such as bones, cartilage, basement membranes, and the like. COL1A1 may be associated with collagen, which is a structural protein distributed in the basement membrane of epithelial tissues and involved in supporting skin cells, and the like. COL1A1 is shown to be overexpressed in all cell groups, which may mean that all cultured skin cells may produce collagen and resulting cytokeratin.

KRT10 is a marker specifically expressed in keratinocytes and may be associated with keratin, which is a scleroprotein involved in the formation of epithelial structures such as hair, nails, and skin. KRT10 is shown to be overexpressed in all cell groups, which may mean that all cultured skin cells may produce keratin and resulting cytokeratin.

KRT7 and KRT19 are markers specifically expressed in keratinocytes and may be associated with keratin, which is a scleroprotein involved in the formation of epithelial structures such as hair, nails, and skin. KRT7 and KRT19 are shown to be overexpressed in group 1, which may mean that cells in group 1 may produce (form) epithelial structures containing keratinocytes of a keratin component.

HS3ST6 may be associated with dermis, extracellular proteoglycan, glycoprotein, and the like. HS3ST6 is shown to be overexpressed in group 1, which may mean that cells in group 1 may produce a component for at least one of dermal cells, proteoglycans and glycoproteins.

CD34 is a marker expressed in various cells, including vascular and skin cells, and may be associated with a transmembrane protein. CD34 is shown to be overexpressed in groups 2 and 3, which may mean that cells in groups 2 and 3 may produce transmembrane proteins and related substances thereto, and further form blood vessels and the like in the skin.

APOE may be associated with apolipoprotein, one of the components of keratinocytes. APOE is shown to be overexpressed in groups 2 and 3, which may mean that all cells in groups 2 and 3 may produce (form) apolipoprotein, one of the components of keratinocytes, and a fat layer associated with the dermal layer.

PECAM1 is a marker expressed in dermal, vascular, and endothelial cells and may be associated with epithelial cells. PECAM1 is shown to be overexpressed in groups 2 and 3, which may mean that cells in groups 2 and 3 may produce (form) epithelial cells and structures such as vascular and endothelial cells in the skin.

That is, the primary skin cells used in the method for producing the skin organoid according to one embodiment of the present disclosure may refer to a heterogeneous cell group expressing at least one of VIM, COL1A1, KRT10, KRT7, KRT19, HS3ST6, CD34, APOE, and PECAM1. Furthermore, organoids, which mimic biological organs, are analogues containing various heterogeneous cells. Accordingly, the primary skin cells of the present disclosure capable of expressing heterogeneous proteins may form an analogue (organoid) similar to an in vivo skin by expressing various heterogeneous cells.

Ultimately, the method for producing the skin organoid according to one embodiment of the present disclosure aims to produce the skin organoid including a similar structure to the biological skin, that is, an epidermis including a stratum corneum as a skin barrier, a dermis including blood vessels and hair roots, and a subcutaneous tissue full skin including fat and the like. Thus, in the skin cells used in the method for producing the skin organoid according to one embodiment of the present disclosure, the primary skin cells of the present disclosure capable of expressing constituent proteins for the epidermis, dermis, and subcutaneous tissue may be most preferable for the formation of the skin organoid.

Referring back to FIG. 1, the first culturing step may mean a step of culturing the primary skin cells to form a first skin organoid including a stratum corneum.

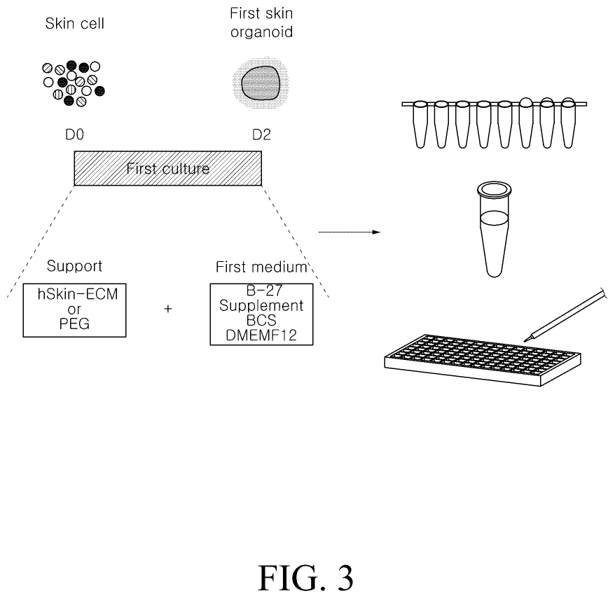

More specifically, referring to FIG. 3, an exemplary diagram for a process of the first culturing step in the method for producing the skin organoid according to one embodiment of the present disclosure is illustrated.

In the first culturing step of the present disclosure, the primary skin cells may be cultured in the first medium with the support.

At this time, the first medium may be a medium containing at least one of a B-27 supplement and a serum replacement.

The B-27 supplement is a growth factor that may improve cell viability and maturity, and in the method for producing the skin organoid according to one embodiment of the present disclosure, the B-27 supplement may be contained in an amount of 1 to 10 v/v % with respect to the total volume of the first medium, but is not limited thereto.

The serum replacement may mean a chemically-defined serum replacement that may promote cell growth, and includes chemically defined components. At this time, in the method for producing the skin organoid according to one embodiment of the present disclosure, the serum replacement may be a commercially available serum replacement, and may be contained in an amount of 5 to 15 v/v % with respect to the total volume of the first medium, but is not limited thereto, and preferably may be 10 v/v %. Accordingly, in the method for producing the skin organoid according to one embodiment of the present disclosure, since the chemically-defined serum replacement is included, the limitation of inducing undesired stimulation by an unclear component such as conventional FBS may be overcome, and the cells may be cultured more stably.

However, the present disclosure is not limited thereto, and the serum replacement in the first medium of the present disclosure may be an animal-derived substance such as bovine calf serum (BCS) as well as the chemically-defined serum replacement. For example, the BCS refers to bovine calf serum, and is serum collected from 16-month-old calves and containing various antibodies and hormones necessary for cell expansion and growth. In the method for producing the skin organoid according to one embodiment of the present disclosure, the BCS may be 5 to 15 v/v % with respect to the total volume of the first medium, but is not limited thereto, and may preferably be 10 v/v %. Meanwhile, in the method for producing the skin organoid according to one embodiment of the present disclosure, as a composition of the first medium, various sera may be used in addition to BCS. For example, the first medium of the present disclosure may include at least one of fetal bovine serum (FBS), dialyzed fetal bovine serum, newborn calf serum (NCS), iron supplemented bovine serum, charcoal stripped serum, de-lipidated serum, human serum, horse serum, and adult bovine serum.

However, a preferred serum used in the first medium of the method for producing the skin organoid according to one embodiment of the present disclosure may be a chemically-defined serum replacement.

Furthermore, the first medium may include a natural or artificial serum-free medium without containing insulin, which is used for maintaining and growing animal cells, as a basic medium, and may include various serum-free media and variants thereof, such as insulin-free minimal essential medium (MEM), Eagle's MEM, Dulbecco's modified Eagle's medium (DMEM), Ham's F12, SF 12, and RPMI 1640, and preferably, DMEM/F12 in which insulin is not contained and DMEM and Ham's F12 are mixed at a ratio of 1:1, but is not limited thereto.

Furthermore, in the first culturing step of the present disclosure, a support may be used, and the support may be an extracellular matrix (ECM) or PEG (polyethylene glycol), but is not limited thereto, and various shapes of supports may be used. For example, the ECM may include at least one of a patch-shaped ECM and a fiber-shaped ECM, and the fiber-shaped ECM may be obtained by treating the patch-shaped ECM with a protein digestive enzyme.

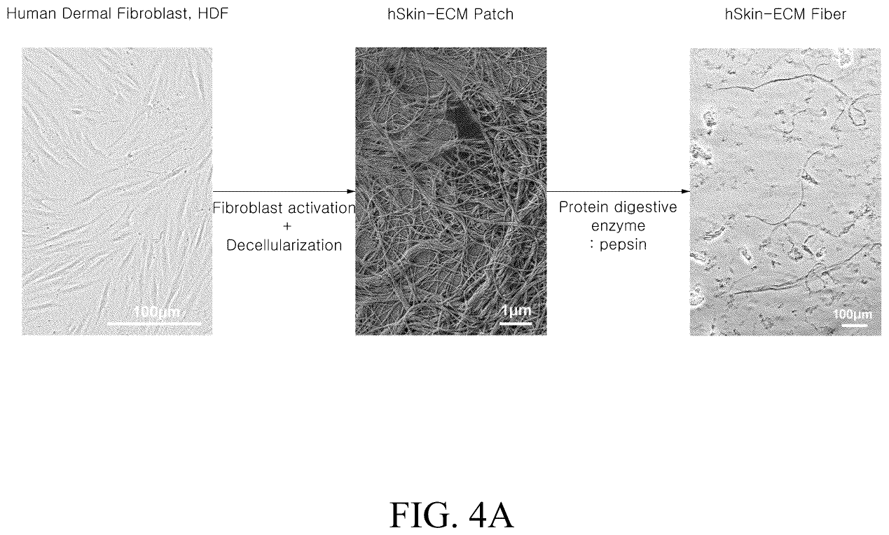

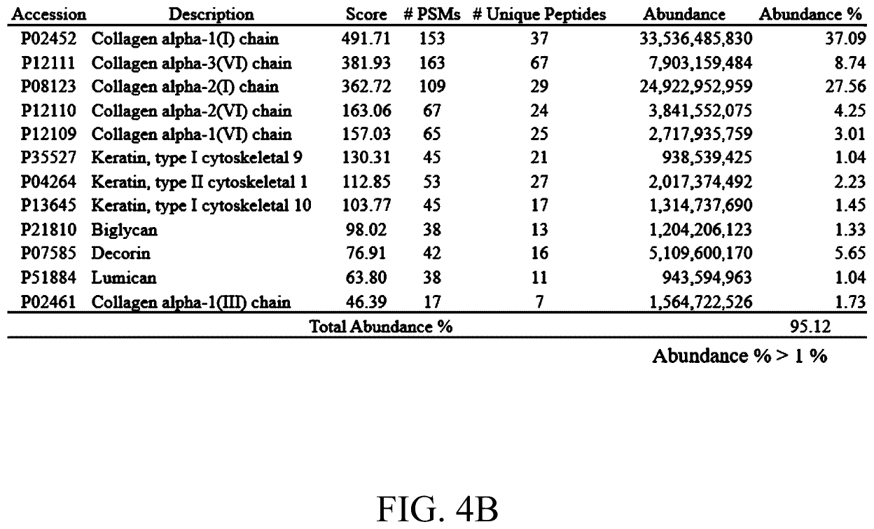

More specifically, referring to FIG. 4A, an exemplary diagram for a process of forming an ECM used in the method for producing the skin organoid according to one embodiment of the present disclosure is illustrated.

The ECM of the present disclosure may be obtained from fibroblasts. For example, in the method for producing the skin organoid according to one embodiment of the present disclosure, the ECM may be obtained by culturing human dermal fibroblasts (HDFs) so that the fibroblasts are activated to form a patch, and then decellularizing the formed fibroblast patch.

The ECM obtained by decellularizing the fibroblast patch may be used in the first culturing step of the present disclosure in the form of a patch with a mesh structure (hSkin-ECM patch). Furthermore, the patch-shaped ECM may be converted into a fiber-shaped ECM by treatment with a protein digestive enzyme, and the fiber-shaped ECM may also be used in the first culturing step of the present disclosure. At this time, pepsin may be used as the protein digestive enzyme, but is not limited thereto, and various digestive enzymes capable of decomposing proteins may be used.

The patch-shaped ECM and the fiber-shaped ECM may be freely selected and used in the method for producing the skin organoid according to one embodiment of the present disclosure, but preferably, the fiber-shaped ECM may be selected and used.

More specifically, referring to FIG. 4B, results of protein component analysis for fiber-shaped ECM used in the method for producing the skin organoid according to one embodiment of the present disclosure are illustrated. At this time, the protein component analysis was performed by liquid chromatography-mass spectrometry (LC-MS).

The fiber-shaped ECM of the present disclosure is shown to include collagen alpha-1(I) chain, collagen alpha-3(VI) chain, collagen alpha-2(I) chain, collagen alpha-2(VI) chain, collagen alpha-1(VI) chain, keratin type I cytoskeletal 9, keratin type II cytoskeletal 1, keratin type I cytoskeletal 10, biglycan, decorin, lumican, and collagen alpha-1(III) chain.

These proteins are scleroproteins associated with the connective tissue of the skin, and all are found to have a high content of 1% or more, accounting for 95.12% of the total ECM. These fiber-shaped ECMs containing a high content of 12 specific proteins may have different compositions from patch-shaped ECMs due to a protein digestive enzyme, and thus, the fiber-shaped ECMs may contain a large surface area and differentiated specific protein compositions to more rapidly form the organoid from the skin cells.

Therefore, the ECM used in the first culturing step of the method for producing the skin organoid according to one embodiment of the present disclosure may preferably be a fiber-shaped ECM, but is not limited thereto, and may be freely selected and used from a patch shape or a shape in which the patch-shaped and fiber-shaped ECMs are mixed.

Referring back to FIG. 3, in general, the direction of expansion and growth of cells is determined by the components of the culture medium and the ECM (extracellular matrix) similar to in vivo conditions, and the growth into an organoid is enabled in a desired direction only when an environment with specific conditions is created. Accordingly, the method for producing the skin organoid according to one embodiment of the present disclosure may form a first skin organoid structurally similar to in vivo skin by including the first culture medium having a specific composition and concentration and the specific-shaped ECM as described above.

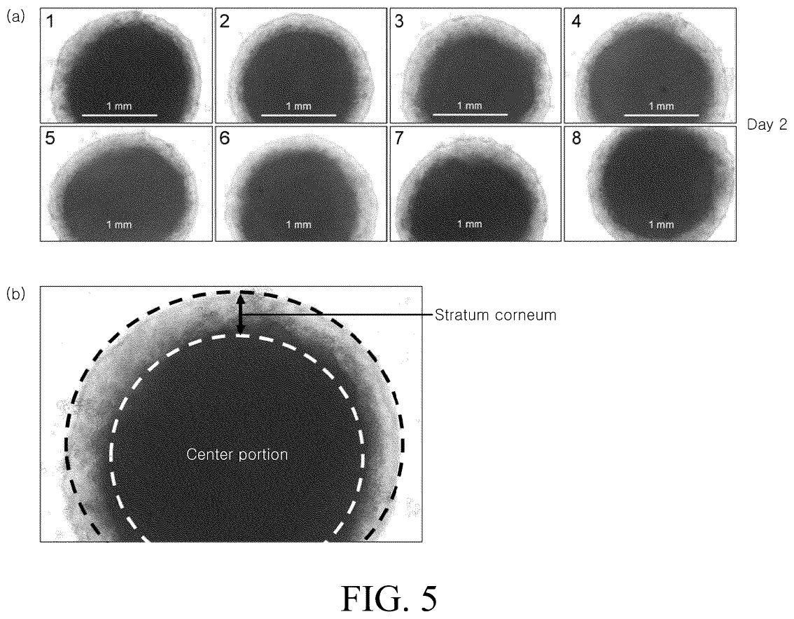

The first skin organoid formed by the first culturing step of the present disclosure may include a center portion (core) which is densely packed with cells and a stratum corneum formed on the outside of the center portion. More specifically, referring to FIG. 5, microscopic images of a first skin organoid formed by the method for producing the skin organoid according to one embodiment of the present disclosure are illustrated.

The first skin organoid of the present disclosure has a round, spherical, three-dimensional shape and may include a stratum corneum with a relatively low density at the outermost side. In addition, the first skin organoid of the present disclosure may have a layered structure and include a center portion (core) which is densely packed with cells below the stratum corneum. At this time, the stratum corneum of the first skin organoid of the present disclosure may mean an epidermis layer including a stratum corneum.

The layered structure of the first skin organoid is shown to be very similar to that of a biological skin, with the stratum corneum present on the outside, the epidermis present below the stratum corneum, and the dermis present on the inside.

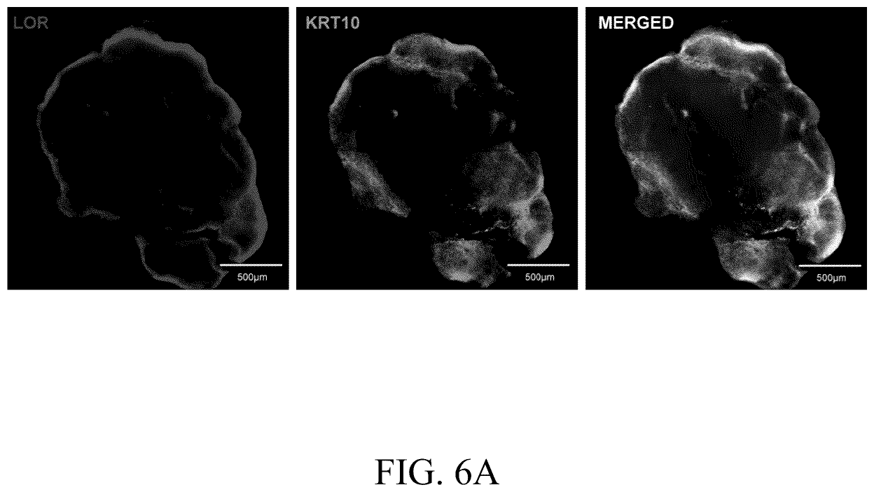

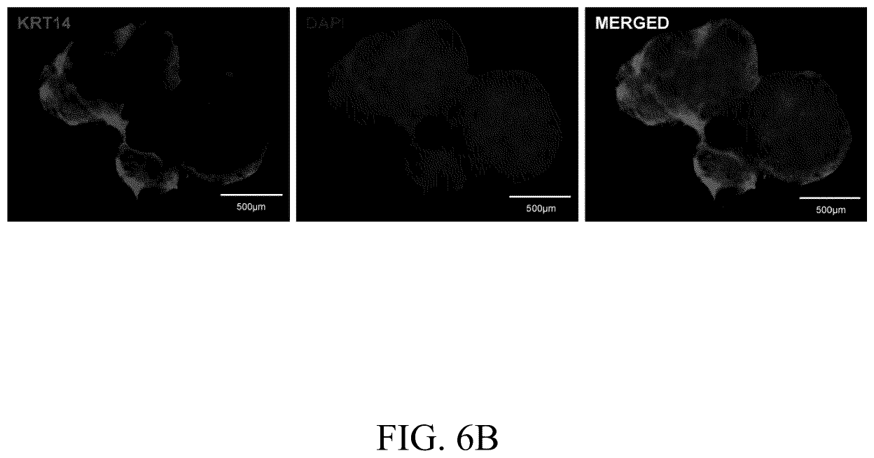

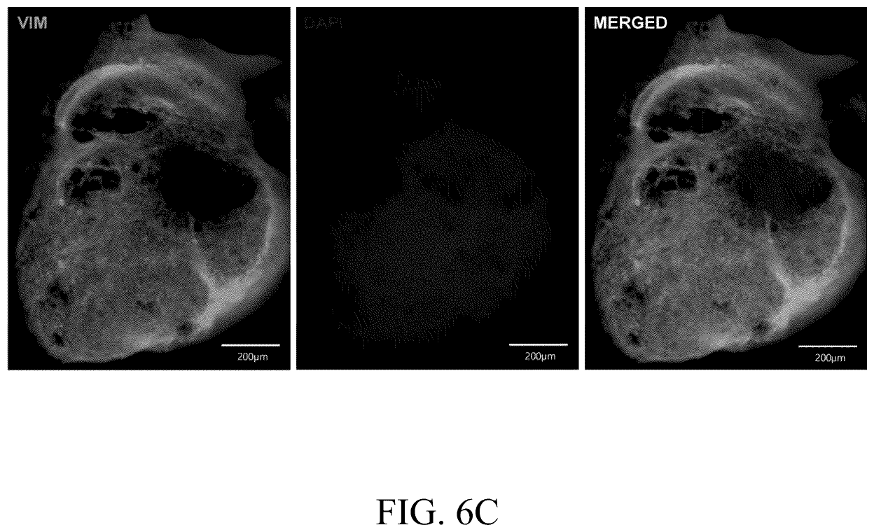

More specifically, referring to FIG. 6A, staining images for LOR and KRT10 markers of a first skin organoid formed by the method for producing the skin organoid according to one embodiment of the present disclosure are illustrated. Loricrin and keratin type I cytoskeletal 10, proteins that constitute the stratum corneum of the skin, are shown to be expressed in the outermost side of the first skin organoid. That is, the first skin organoid of the present disclosure includes a stratum corneum in the outermost side, i.e., the skin, which may mean that the first skin organoid has a structure similar to a biological skin.

Furthermore, referring to FIG. 6B, staining images for a KRT14 marker of the first skin organoid formed by the method for producing the skin organoid according to one embodiment of the present disclosure are illustrated. KRT14, a marker for keratinocytes and epidermal cells, is shown to be expressed on the surface of the first skin organoid, i.e., below the LOR and KRT10 marker expression layer in FIG. 6A described above. That is, the first skin organoid of the present disclosure includes a stratum corneum and an epidermal layer on the surface, which may mean that the first skin organoid has a structure similar to a biological skin.

Furthermore, referring to FIG. 6C, staining images for a VIM marker of the first skin organoid formed by the method for producing the skin organoid according to one embodiment of the present disclosure are illustrated. VIM, a marker for fibroblasts in the skin, i.e., the dermis, is shown to be expressed inside the first skin organoid, i.e., below the KRT14 marker expression layer in FIG. 6B described above. That is, the first skin organoid of the present disclosure includes a center portion corresponding to a dermal layer inside, which may mean that the first skin organoid has a structure similar to a biological skin.

Therefore, the first skin organoid formed by the method for producing the skin organoid according to one embodiment of the present disclosure is a three-dimensional skin organoid that structurally closely mimics a biological skin.

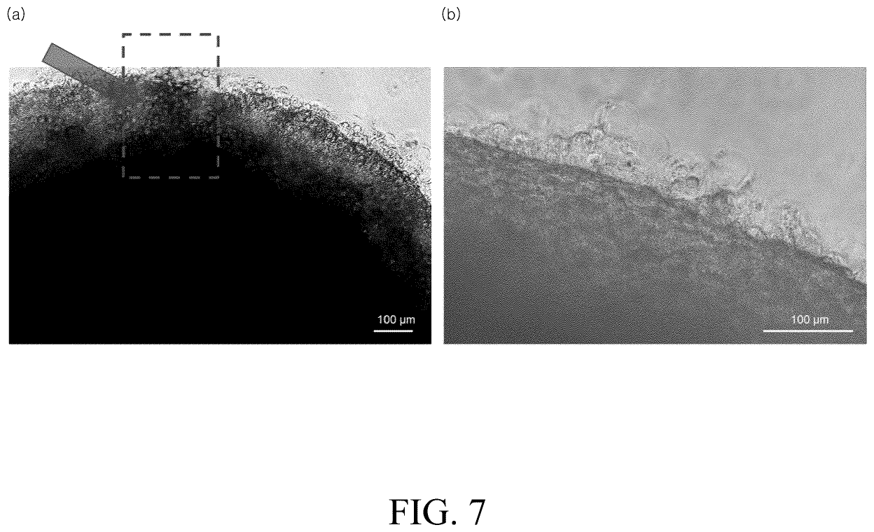

Furthermore, the first skin organoid of the present disclosure may also be physiologically very similar to a biological skin. More specifically, referring to FIG. 7, microscopic images of the stratum corneum of the first skin organoid formed by the method for producing the skin organoid according to one embodiment of the present disclosure are illustrated.

Referring to (a) of FIG. 7, cells distributed on the boundary of the center portion of the first skin organoid may move to the outermost stratum corneum of the organoid, and referring to (b) of FIG. 7, the moved cells are shown to die and be distributed at the outermost side of the first skin organoid.

The cell migration and the cell death in the outermost layer are shown to be similar to the physiology of skin cells in which epidermal cells in the basal layer are gradually pushed upwards and die when reaching the stratum corneum. That is, the first skin organoid of the present disclosure may mean having a physiological function of a biological skin, such as a skin regeneration cycle, and thus may represent the physiological function of a biological skin.

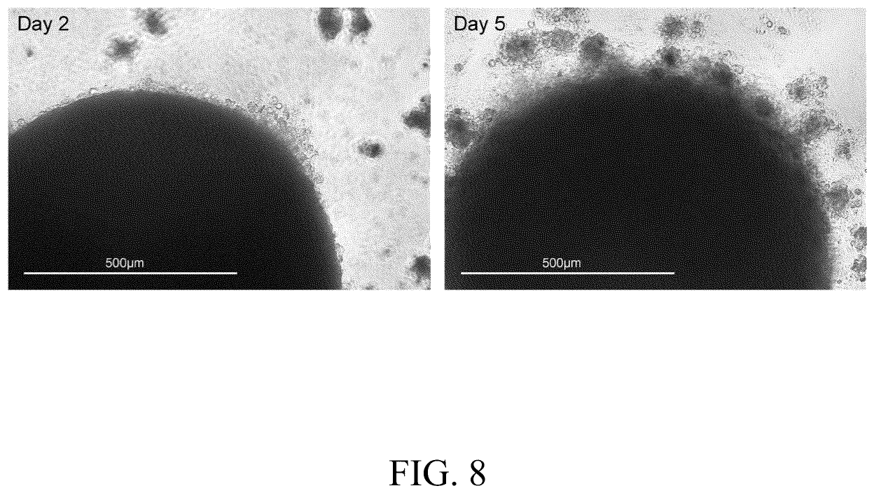

Meanwhile, the first skin organoid of the present disclosure may have a size of about 100 to 3000 μm. More specifically, referring to FIG. 8, microscopic images of the size of the first skin organoid formed by the method for producing the skin organoid according to one embodiment of the present disclosure are illustrated.

The central portion of the first skin organoid of the present disclosure is shown to have a diameter of 500 μm or more when cultured for 2 days, and the stratum corneum is shown to have a thickness of about 100 μm or less.

Furthermore, when the first skin organoid of the present disclosure is cultured for 5 days, the diameter of the center portion is shown to be longer than that on day 2, and the thickness of the stratum corneum is shown to be thicker than that on day 2.

That is, the first skin organoid of the present disclosure may be cultured for 3 days or more and grow continuously. However, the first skin organoid formed by the method for producing the skin organoid according to one embodiment of the present disclosure may preferably include a center portion with a diameter of about 100 to 1000 μm and a stratum corneum with a thickness of 1 to 100 μm or less.

More specifically, referring back to FIG. 3, the first skin organoid having the size described above may be formed by culturing for a period of the first culturing step, that is, at least one period of about 1 hour to 3 days, and may have a size of about 1,100 μm in diameter, including a center portion with a diameter of about 100 to 1,000 μm and a stratum corneum with a thickness of 1 to 100 μm or less, but is not limited thereto.

Accordingly, the first skin organoid of the aforementioned size may be stored and used in various commercially available cell containers such as a 96-well plate and a micro tube, with a size of about 1,100 μm or less.

Ultimately, the first skin organoid of the present disclosure may be formed from 1 hour after the start of the first culture, and the first culturing step for forming the first skin organoid may be a step of culturing for at least one period of about 1 hour to 3 days, but the most preferable culturing period may be a period of about 2 days (about 48 hours).

Meanwhile, the first skin organoid of the present disclosure may preferably have a size of about 1,100 μm or less, which is easy to be stored in cells, for commercial use or for easy storage, but is not limited thereto, and the first skin organoid of the present disclosure may be cultured for 3 days or more, and thus may have more diverse sizes and additional biomimetic structures.

More specifically, referring back to FIG. 1, the first skin organoid formed through the first culturing step may be cultured into a second skin organoid including a hair root by forming the hair root through the second culturing step. That is, the second culturing step may mean a step of culturing the first skin organoid to form the second skin organoid including the hair root.

At this time, the first skin organoid may be cultured in the second medium. More specifically, the second medium may be a medium containing at least one of the first medium, ascorbic acid, CHIRR99021, and a Wnt agonist.

More specifically, the ascorbic acid is an antioxidant that is involved in procollagen synthesis and is a cofactor associated with increased type 1 collagen production. The ascorbic acid may stimulate and regulate the expansion of various cells such as adipocytes, osteoblasts, and chondrocytes in vitro. Furthermore, when a certain concentration of ascorbic acid is added to the skin cell culture medium, the ascorbic acid acts as a cell growth promoter to increase cell expansion ability, and even promote DNA synthesis. However, when the concentration of ascorbic acid is not appropriate, the ascorbic acid may inhibit the expansion ability of cells and have cytotoxicity to cause apoptosis. Accordingly, the appropriate concentration of ascorbic acid used in the second medium of the present disclosure may be 2 to 500 mM based on 1 L of the second medium, but is not limited thereto, and when ascorbic acid is added at the above-mentioned concentration, the expansion ability of skin organoid cells may be improved.

The CHIRR99021 is a material that inhibits the activity of glycogen synthase kinase (GSK)-3β. More specifically, as GSK-3β is inhibited, β-catenin of a signaling system involved in cell expansion is not decomposed by GSK-3β, and thus, the expression level of genes involved in cell expansion is increased, thereby improving the survival and expansion of cells.

The Wnt agonist is a substance for activating a signaling pathway, and may activate a Wnt protein that contributes to the signaling system within organoid cells to promote a metabolism thereof, thereby more actively promoting the growth of the skin organoid. That is, the size of the skin organoid may be improved by adding the Wnt agonist.

The second culturing step of the present disclosure may be a step of culturing for at least one period of about 7 to 40 days, and from day 7 of the second culture, the first skin organoid grows into a second skin organoid including a hair root.

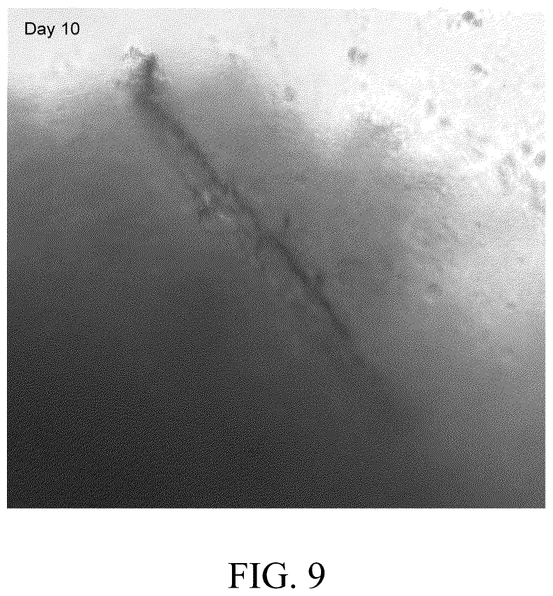

More specifically, referring to FIG. 9, microscopic images of the second skin organoid formed by the method for producing the skin organoid according to one embodiment of the present disclosure are illustrated.

The second skin organoid cultured for 10 days, including the first and second culture steps, is shown to form a hair root-like structure. Furthermore, the second skin organoid is shown to contain hair, as some hair shafts are exposed to the stratum corneum.

That is, the second skin organoid of the present disclosure includes hair roots and hair, and may structurally mimic and represent the skin from which body hair and scalp hair may be formed.

Referring back to FIG. 1, according to the above process, the method for producing the skin organoid according to one embodiment of the present disclosure includes a step of performing a first culture of skin cells in a first medium with at least one support among an extracellular matrix (ECM) and polyethylene glycol (PEG) to form a first skin organoid; and a step of performing a second culture of the first skin organoid in a second medium, thereby forming an organoid including a stratum corneum and a hair root formed on the outside.

More specifically, the method for preparing the skin organoid according to one embodiment of the present disclosure may provide two types of skin organoids depending on the presence or absence of hair roots and hair. The final skin organoid including the hair roots and hair is a skin organoid that is structurally and functionally similar to a biological skin, includes a center portion (core) which is densely packed with cells, a stratum corneum formed on the outside of the center portion, and a hair root, in which the cells can move from the center portion to the stratum corneum, and has a round, spherical, three-dimensional shape.

That is, the method for producing the skin organoid according to one embodiment of the present disclosure may form and provide a biocompatible model having the shape and function very similar to the biological skin.

Meanwhile, the skin organoid formed by the method for producing the skin organoid according to one embodiment of the present disclosure is very morphologically and physiologically similar to the biological skin and thus can provide an experimental result of evaluating more accurate and reliable stability and toxicity drug than conventional skin organoids.

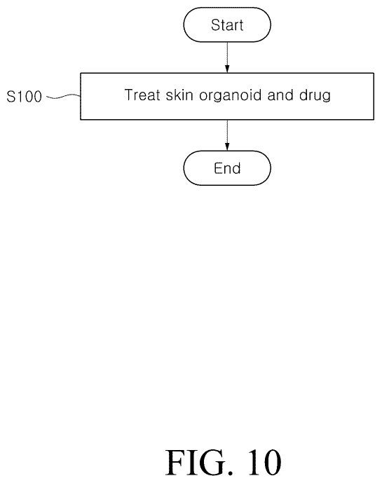

For example, referring to FIG. 10, a flowchart for a method for evaluating a drug using a skin organoid according to one embodiment of the present disclosure is illustrated.

The method for evaluating the drug using the skin organoid according to one embodiment of the present disclosure may include a step (S100) of treating a skin organoid and a drug.

The skin organoid used in the method for evaluating the drug using the skin organoid according to one embodiment of the present disclosure is structurally and functionally (physiologically) similar to the biological skin, includes a center portion (core), which is densely packed with cells, a stratum corneum formed on the outside of the center portion, and a hair root, in which the cells can move from the center portion to the stratum corneum, and has a round, spherical, three-dimensional shape, and thus may be used as a biomimetic model with higher biocompatibility.

That is, the skin organoid of the present disclosure may be used as a human-mimetic model for evaluating side effects, toxicity, and efficacy of drugs, and may also be used for experiments to determine the stability and effects of skin toxicity of candidate substances during the new drug development process.

In this case, the term “drug” as used herein may include all materials used to change or examine a physiological system or disease conditions for the benefit of living things. More specifically, the drug may include at least one selected from the group consisting of vitamins, hormones, metal salts, vaccines, antiserum agents, antibiotics, chemotherapeutic agents, cardiac agents, blood pressure regulators, antihistamines, steroids, antitoxins and contrast agents, but is not limited.

For example, the skin organoid reacts with the drug on a plate, and then the conditions thereof are observed microscopically or changes thereof may be confirmed through protein analysis, etc.

Although the embodiments of the present disclosure have been described in detail with reference to the accompanying drawings, the present disclosure is not limited thereto and may be embodied in many different forms without departing from the technical concept of the present disclosure. Therefore, the embodiments of the present disclosure are provided for illustrative purposes only but not intended to limit the technical concept of the present disclosure. The scope of the technical concept of the present disclosure is not limited thereto. Therefore, it should be appreciated that the aforementioned embodiments are illustrative in all aspects and are not restricted. The protective scope of the present disclosure should be construed on the basis of the appended claims, and all the technical ideas in the equivalent scope thereof should be construed as falling within the scope of the present disclosure.

### National Research and Development Project Supporting the Invention

[Project Unique Number] 1425164801

[Project Number] S3222933

[Department Name] Ministry of SMEs and Startups (MSS)

[Project Management (Special) Institution Name] Korea Technology & Information Promotion Agency for SMEs

[Research Project Name] Startup growth and technological development

[Research Subject Name] Organoid-dedicated one-step tissue clearing solution

[Contribution ratio] 1/1

[Project performance institute name] ORG Co., Ltd.

[Research Period] May 2, 2022 to May 1, 2023

1. A skin organoid comprising:
a center portion (core) which is densely packed with cells;
a stratum corneum formed on an outside of the center portion; and
a hair root,
wherein the cells are able to move from the center portion to the stratum corneum, and
has a round, spherical, three-dimensional shape.

2. The skin organoid of claim 1, wherein the stratum corneum has a thickness of 1 to 100 μm or less.

3. The skin organoid of claim 1, wherein the stratum corneum includes at least one of KRT10, KRT14 and loricrin.

4. The skin organoid of claim 1, wherein the center portion has a diameter of 100 to 1000 μm.

5. The skin organoid of claim 1, wherein the center portion includes vimentin

6. A method for producing a skin organoid including a stratum corneum formed on an outside and a hair root, the method comprising:
performing a first culture of skin cells (primary skin cells) in a first medium with at least one support of an extracellular matrix (ECM) and polyethylene glycol (PEG) to form a first skin organoid; and
performing a second culture of the first skin organoid in a second medium.

7. The method of claim 6, further comprising:
before the performing a first culture,
chopping a skin tissue isolated from a subject; and
Obtaining heterogeneous skin cells (primary skin cells) by treating the chopped skin tissue with collagenase.

8. The method of claim 7, wherein the collagenase includes at least one of type I collagenase and type IV collagenase.

9. The method of claim 7, wherein the obtaining heterogeneous skin cells further includes culturing the primary skin cells to expand.

10. The method of claim 6, wherein the skin cell has a size of about 100 μm or less.

11. The method of claim 6, wherein the skin cell includes at least one of APOE, COL1A1, CD34, KRT7, KRT10, KRT19, HS3ST6, PECAM1 and VIM.

12. The method of claim 6, wherein the ECM includes at least one of a patch-shaped ECM and a fiber-shaped ECM.

13. The method of claim 12, wherein the fiber-shaped ECM is obtained by treating the patch-shaped ECM with a protein digestive enzyme.

14. The method of claim 12, wherein the fiber-shaped ECM includes at least one of collagen alpha-1(I) chain, collagen alpha-3(VI) chain, collagen alpha-2(I) chain, collagen alpha-2(VI) chain, collagen alpha-1(VI) chain, keratin type I cytoskeletal 9, keratin type II cytoskeletal 1, keratin type I cytoskeletal 10, biglycan, decorin, lumican and collagen alpha-1(III) chain.

15. The method of claim 6, wherein the ECM is obtained by culturing fibroblasts to form a fibroblast patch and decellularizing the fibroblast patch.

16. The method of claim 6, wherein the first culture is performed for at least one period of about 1 hour to 3 days.

17. The method of claim 6, wherein the first medium includes at least one of a B-27 supplement and a serum replacement.

18. The method of claim 17, wherein the B-27 supplement is contained in an amount of 1 to 10 v/v % with respect to a total volume of the first medium.

19. The method of claim 17, wherein the serum replacement is contained in an amount of 5 to 15 v/v % with respect to a total volume of the first medium.

20. The method of claim 6, wherein the second culture is performed for at least one period of about 7 days to 40 days.

21. (canceled)

22. (canceled)

23. (canceled)
